(19) United States
(12) Patent Application Publication
(10) Pub. No.: US 2005/0013865 A1
(43) Pub. Date: Jan. 20, 2005

(54) Super-absorbing polymers containing tocopherol

(76) Inventors: Gerhard Nestler, Wien (AT); Jurgen Schroder, Ludwigshafen (DE); Stefan Wickel, Bissersheim (DE)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/496,775

(22) PCT Filed: Dec. 18, 2002
(86) PCT No.: PCT/EP02/14451

(30) Foreign Application Priority Data
Dec. 21, 2001 (DE) 10163543.5

(51) Int. Cl. A61K 31/355; A61K 9/14

(52) U.S. Cl. 424/487; 514/458

(57) ABSTRACT

The present invention relates to novel tocopherol-containing superabsorbents, to a process for preparing them using tocopherol-containing acid-functional monomers, especially acrylic acid, and to their use for absorbing aqueous fluids.

The present invention relates to novel tocopherol-containing superabsorbents, to a process for preparing them using tocopherol-containing acid-functional monomers, especially acrylic acid, and to their use for absorbing aqueous fluids.

More particularly, the present invention relates to highly swellable hydrogels (superabsorbents) based on polyacrylic acid which contain alpha-tocopherol in particular, to processes for preparing them using alpha-tocopherol-containing acrylic acid and also to their use in a hygiene article.

Swellable hydrogel-forming addition polymers, known as superabsorbent polymers or SAPs, are known from the prior art. They are networks of flexible hydrophilic addition polymers, which can be both ionic and nonionic in nature. They are capable of absorbing and binding aqueous fluids by forming a hydrogel and therefore are preferentially used for manufacturing tampons, diapers, sanitary napkins, incontinence articles, training pants for children, insoles and other hygiene articles for the absorption of body fluids. Within hygiene articles, superabsorbents are generally positioned in an absorbent core which, as well as SAP, comprises other materials, including fibers (cellulose fibers), which act as a kind of liquid buffer to intermediately store the spontaneously applied liquid insults and are intended to ensure efficient channelization of the body fluids in the absorbent core toward the superabsorbent.

The current trend in diaper design is toward ever thinner constructions having a reduced cellulose fiber content and an increased hydrogel content. The trend toward ever thinner diaper constructions has substantially changed the performance profile required of the water swellable hydrophilic polymers over the years. Whereas at the start of the development of highly absorbent hydrogels it was initially solely the very high swellability on which interest focused, it was subsequently determined that the ability of the superabsorbent to transmit and distribute fluid is also of decisive importance. It has been determined that superabsorbents greatly swell at the surface on wetting with liquid, so that transportation of liquid into the particle interior is substantially compromised or completely prevented. This trait of superabsorbents is known as gel-blocking. The greater amount of polymer per unit area in the hygiene article must not cause the swollen polymer to form a barrier layer to subsequent fluid. A product having good transportation properties will ensure optimal utilization of the entire hygiene article.

Superabsorbent hydrogels for use in the hygiene sector are at present addition polymers having a degree of neutralization in the range from 5 to 80 mol %, especially in the range from 60 to 80 mol %, based on the polymerized acid-functional monomer units.

Appropriate transportation properties are possessed for example by hydrogels having high gel strength in the swollen state. Gels lacking in strength are deformable under an applied pressure, for example pressure due to the bodyweight of the wearer of the hygiene article, and clog the pores in the SAP/cellulose fiber absorbent and so prevent continued absorption of fluid. Enhanced gel strength is generally obtained through a higher degree of crosslinking, although this reduces retention performance. An elegant way to enhance gel strength is surface postcrosslinking. In this process, dried superabsorbents having an average crosslink density are subjected to an additional crosslinking step. The process is known to one skilled in the art and described in EP-A-0 349 240. Surface postcrosslinking increases the crosslink density in the sheath of the superabsorbent particle, whereby the absorbency under load is raised to a higher level. Whereas the absorption capacity decreases in the superabsorbent sheath, the core has an improved absorption capacity (compared to the sheath) owing to the presence of mobile polymer chains, so that sheath construction ensures improved fluid transmission without occurrence of the gel-blocking effect. It is perfectly desirable for the total capacity of the superabsorbent to be occupied not spontaneously but with time delay. Since the hygiene article is generally repeatedly insulted with urine, the absorption capacity of the superabsorbent should sensibly not be exhausted after the first disposition. Ideally, the superabsorbent will continue to absorb the fluid speedily even on further disposition. In any case, a surge of fluid or a further second or subsequent insult may cause reemergence of the fluid and hence rewet. However, when the phenomenon of gel-blocking occurs, this may cause the fluid to leak from the hygiene article. This fluid will also contain soluble components of the superabsorbent. Rewet is reduced by modern superabsorbents, but not completely prevented.

It is an object of the present invention to provide modified superabsorbents and a process for making them which on use in hygiene articles, for example, ideally cannot lead to any potential health hazards due to the occurrence of rewet. It is another object of the present invention to improve the process for preparing superabsorbents.

We have found that these objects are achieved, surprisingly, by the use of highly swellable hydrogels which are polymerized from an acrylic acid which has been admixed with tocopherol.

The present invention accordingly provides hydrogel-forming polymers capable of absorbing aqueous fluids and based on polyacrylate which contain tocopherol. Preference is given to hydrogel-forming polymers capable of absorbing aqueous fluids where the tocopherol is distributed over the polymer. Preference is further given to hydrogel-forming polymers capable of absorbing aqueous fluids where the tocopherol is alpha-tocopherol. Especially such hydrogel-forming polymers capable of absorbing aqueous fluids where the tocopherol is present in a range from 10 to 1 000 ppm based on acid-functional monomers, especially acrylic acid or acrylate.

The present invention further provides processes for preparing hydrogel-forming polymers capable of absorbing aqueous fluids and based on polymers which bear acid groups, especially based on polyacrylate, by polymerizing acid-functional monomers, especially acrylic acid, which contain tocopherol. Preference is given to processes where the tocopherol is alpha-tocopherol and where tocopherol is present in a range from 10 to 1 000 ppm based on acrylic acid.

The present invention further provides hydrogel-forming polymers capable of absorbing fluid and based on acid-functional polymers, especially polyacrylate, obtainable by the above processes and their use for absorbing aqueous fluids, especially in hygiene articles.

The present invention also provides acid-functional monomer, especially acrylic acid, containing tocopherol, especially alpha-tocopherol. The present invention further provides acid-functional monomer, especially acrylic acid, containing tocopherol stabilizer. Tocopherol preferably makes up at least 50 mol % of the stabilizer, particularly preferably at least 90 mol % of the stabilizer, and especially only tocopherol stabilizer is used. The present invention also provides for the use of tocopherol to stabilize acid-functional monomers, especially acrylic acid and salts thereof.

Experimental Part

Methods of Making

a) Monomers Used

Hydrogel-forming polymers are in particular polymers of (co)polymerized hydrophilic monomers, graft (co)polymers of one or more hydrophilic monomers on a suitable grafting base, crosslinked cellulose or starch ethers, crosslinked carboxymethylcellulose, partially crosslinked polyalkylene oxide or natural products that swell in aqueous fluids, for example guar derivatives, alginates and carrageenans.

Suitable grafting bases can be of natural or synthetic origin. Examples are starch, cellulose or cellulose derivatives and also other polysaccharides and oligosaccharides, polyvinyl alcohol, polyalkylene oxides, especially polyethylene oxides and polypropylene oxides, polyamines, polyamides and also hydrophilic polyesters. Suitable polyalkylene oxides have for example the formula 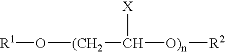
where
R¹ and R² are independently hydrogen, alkyl, alkenyl or acryl,
X is hydrogen or methyl and
n is an integer from 1 to 10 000.

R¹ and R² are each preferably hydrogen, (C₁-C₄)-alkyl, (C₂-C₆)-alkenyl or phenyl.

Preferred hydrogel-forming polymers are crosslinked polymers having acid groups, which are predominantly in the form of their salts, generally alkali metal or ammonium salts. Such polymers swell particularly strongly on contact with aqueous fluids to form gels.

Preference is given to polymers which are obtained by crosslinking polymerization or copolymerization of acid-functional monoethylenically unsaturated monomers or salts thereof. It is further possible to (co)polymerize these monomers without crosslinker and to crosslink them subsequently.

Examples of such monomers bearing acid groups are monoethylenically unsaturated C₃- to C₂₅-carboxylic acids or anhydrides such as acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. It is also possible to use monoethylenically unsaturated sulfonic or phosphonic acids, for example vinylsulfonic acid, allylsulfonic acid, sulfoethyl acrylate, sulfomethyl acrylate, sulfopropyl acrylate, sulfopropyl methacrylate, 2-hydroxy-3-acryloyloxypropylsulfonic acid, 2-hydroxy-3-methacryloyloxypropylsulfonic acid, vinylphosphonic acid, allylphosphonic acid, styrenesulfonic acid and 2-acrylamido-2-methylpropanesulfonic acid. The monomers may be used alone or mixed.

Preferred monomers are acrylic acid, methacrylic acid, vinylsulfonic acid, acrylamidopropanesulfonic acid or mixtures thereof, for example mixtures of acrylic acid and methacrylic acid, mixtures of acrylic acid and acrylamidopropanesulfonic acid or mixtures of acrylic acid and vinylsulfonic acid. Particular preference is given to acrylic acid. When, in the present invention, concentrations are defined on the basis of acid-functional monomers, these concentrations relate to the totality of monomers irrespective of whether the acid group is present in protonated or deprotonated form. Where the concentration relates to acid group containing polymers, the concentration will relate to the level of acid groups in protonated and deprotonated form.

To optimize properties, it can be sensible to use additional monoethylenically unsaturated compounds which do not bear an acid group but are copolymerizable with the monomers bearing acid groups. Such compounds include for example the amides and nitriles of monoethylenically unsaturated carboxylic acid, for example acrylamide, methacrylamide and N-vinylformamide, N-vinylacetamide, N-methyl-N-vinylacetamide, acrylonitrile and methacrylonitrile. Examples of further suitable compounds are vinyl esters of saturated C₁- to C₄-carboxylic acids such as vinyl formate, vinyl acetate or vinyl propionate, alkyl vinyl ethers having at least 2 carbon atoms in the alkyl group, for example ethyl vinyl ether or butyl vinyl ether, esters of monoethylenically unsaturated C₃- to C₆-carboxylic acids, for example esters of monohydric C₁- to C₁₈-alcohols and acrylic acid, methacrylic acid or maleic acid, monoesters of maleic acid, for example methyl hydrogen maleate, N-vinyllactams such as N-vinylpyrrolidone or N-vinylcaprolactam, acrylic and methacrylic esters of alkoxylated monohydric saturated alcohols, for example of alcohols having from 10 to 25 carbon atoms which have been reacted with from 2 to 200 mol of ethylene oxide and/or propylene oxide per mole of alcohol, and also monoacrylic esters and monomethacrylic esters of polyethylene glycol or polypropylene glycol, the molar masses (Mn) of the polyalkylene glycols being up to 2 000, for example. Further suitable monomers are styrene and alkyl-substituted styrenes such as ethylstyrene or tert-butylstyrene.

These monomers without acid groups may also be used in mixture with other monomers, for example mixtures of vinyl acetate and 2-hydroxyethyl acrylate in any proportion. These monomers without acid groups are added to the reaction mixture in amounts within the range from 0 to 50% by weight, preferably less than 20% by weight.

Preference is given to crosslinked polymers of monoethylenically unsaturated monomers which bear acid groups and which are optionally converted into their alkali metal or ammonium salts before or after polymerization and of 0-40% by weight, based on their total weight, of monoethylenically unsaturated monomers which do not bear acid groups.

Preference is given to crosslinked polymers of monoethylenically unsaturated C₃- to C₁₂-carboxylic acids and/or their alkali metal or ammonium salts. Preference is given in particular to crosslinked polyacrylic acids where 5-80 mol %, preferably 60-80 mol % of the acid groups, and in the case of acidic superabsorbents 5-30 mol %, preferably 5-20 mol %, particularly preferably 5-10 mol % based on the monomers containing acid groups, are present as alkali metal or ammonium salts.

Possible crosslinkers include compounds containing at least two ethylenically unsaturated double bonds. Examples of compounds of this type are N,N′-methylenebisacrylamide, polyethylene glycol diacrylates and polyethylene glycol dimethacrylates each derived from polyethylene glycols having a molecular weight of from 106 to 8 500, preferably from 400 to 2 000, trimethylolpropane triacrylate, trimethylolpropane trimethacrylate, ethylene glycol diacrylate, ethylene glycol dimethacrylate, propylene glycol diacrylate, propylene glycol dimethacrylate, butanediol diacrylate, butanediol dimethacrylate, hexanediol diacrylate, hexanediol dimethacrylate, allyl methacrylate, diacrylates and dimethacrylates of block copolymers of ethylene oxide and propylene oxide, polyhydric alcohols, such as glycerol or pentaerythritol, doubly or more highly esterified with acrylic acid or methacrylic acid, triallylamine, dialkyldiallylammonium halides such as dimethyldiallylammonium chloride and diethyldiallylammonium chloride, tetraallylethylenediamine, divinylbenzene, diallyl phthalate, polyethylene glycol divinyl ethers of polyethylene glycols having a molecular weight of from 106 to 4 000, trimethylolpropane diallyl ether, butanediol divinyl ether, pentaerythritol triallyl ether, reaction products of 1 mol of ethylene glycol diglycidyl ether or polyethylene glycol diglycidyl ether with 2 mol of pentaerythritol triallyl ether or allyl alcohol, and/or divinylethyleneurea. Preference is given to using water-soluble crosslinkers, for example N,N′-methylenebisacrylamide, polyethylene glycol diacrylates and polyethylene glycol dimethacrylates derived from addition products of from 2 to 400 mol of ethylene oxide with 1 mol of a diol or polyol, vinyl ethers of addition products of from 2 to 400 mol of ethylene oxide with 1 mol of a diol or polyol, ethylene glycol diacrylate, ethylene glycol dimethacrylate or triacrylates and trimethacrylates of addition products of from 6 to 20 mol of ethylene oxide with 1 mol of glycerol, pentaerythritol triallyl ether and/or divinylurea.

Possible crosslinkers also include compounds containing at least one polymerizable ethylenically unsaturated group and at least one further functional group. The functional group of these crosslinkers has to be capable of reacting with the functional groups, essentially the acid groups, of the monomers. Suitable functional groups include for example hydroxyl, amino, epoxy and aziridino groups. Useful are for example hydroxyalkyl esters of the abovementioned monoethylenically unsaturated carboxylic acids, e.g., 2-hydroxyethyl acrylate, hydroxypropyl acrylate, hydroxybutyl acrylate, hydroxyethyl methacrylate, hydroxypropyl methacrylate and hydroxybutyl methacrylate, allylpiperidinium bromide, N-vinylimidazoles, for example N-vinylimidazole, 1-vinyl-2-methylimidazole and N-vinylimidazolines such as N-vinylimidazoline, 1-vinyl-2-methylimidazoline, 1-vinyl-2-ethylimidazoline or 1-vinyl-2-propylimidazoline, which can be used in the form of the free bases, in quaternized form or as salt in the polymerization. It is also possible to use dialkylaminoethyl acrylate, dimethylaminoethyl methacrylate, diethylaminoethyl acrylate and diethylaminoethyl methacrylate. The basic esters are preferably used in quaternized form or as salt. It is also possible to use glycidyl(meth)acrylate, for example.

Useful crosslinkers further include compounds containing at least two functional groups capable of reacting with the functional groups, essentially the acid groups, of the monomers. Suitable functional groups were already mentioned above, i.e., hydroxyl, amino, epoxy, isocyanato, ester, amido and aziridino groups. Examples of such crosslinkers are ethylene glycol, diethylene glycol, triethylene glycol, tetraethylene glycol, polyethylene glycol, glycerol, polyglycerol, triethanolamine, propylene glycol, polypropylene glycol, block copolymers of ethylene oxide and propylene oxide, ethanolamine, sorbitan fatty acid esters, ethoxylated sorbitan fatty acid esters, trimethylolpropane, pentaerythritol, 1,3-butanediol, 1,4-butanediol, polyvinyl alcohol, sorbitol, starch, polyglycidyl ethers such as ethylene glycol diglycidyl ether, polyethylene glycol diglycidyl ether, glycerol diglycidyl ether, glycerol polyglycidyl ether, diglycerol polyglycidyl ether, polyglycerol polyglycidyl ether, sorbitol polyglycidyl ether, pentaerythritol polyglycidyl ether, propylene glycol diglycidyl ether and polypropylene glycol diglycidyl ether, polyaziridine compounds such as 2,2-bishydroxymethylbutanol tris[3-(1-aziridinyl)propionate], 1,6-hexamethylenediethyleneurea, diphenylmethanebis-4,4′-N,N′-diethyleneurea, haloepoxy compounds such as epichlorohydrin and α-methylepifluorohydrin, polyisocyanates such as 2,4-toluylene diisocyanate and hexamethylene diisocyanate, alkylene carbonates such as 1,3-dioxolan-2-one and 4-methyl-1,3-dioxolan-2-one, also bisoxazolines and oxazolidones, polyamidoamines and also their reaction products with epichlorohydrin, also polyquaternary amines such as condensation products of dimethylamine with epichlorohydrin, homo- and copolymers of diallyldimethylammonium chloride and also homo- and copolymers of dimethylaminoethyl (meth)acrylate which are optionally quaternized with, for example, methyl chloride.

Useful crosslinkers further include multivalent metal ions capable of forming ionic crosslinks. Examples of such crosslinkers are magnesium, calcium, barium and aluminum ions. These crosslinkers are used for example as hydroxides, carbonates or bicarbonates. Useful crosslinkers further include multifunctional bases likewise capable of forming ionic crosslinks, for example polyamines or their quaternized salts. Examples of polyamines are ethylenediamine, diethylenetriamine, triethylenetetramine, tetraethylenepentamine, pentaethylenehexamine and polyethyleneimines and also polyamines having molar masses in each case of up to 4 000 000.

The crosslinkers are present in the reaction mixture for example from 0.001 to 20% and preferably from 0.01 to 14% by weight.

b) Free Radical Polymerization

The polymerization is initiated in the generally customary manner, by means of an initiator. But the polymerization may also be initiated by electron beams acting on the polymerizable aqueous mixture. However, the polymerization may also be initiated in the absence of initiators of the above-mentioned kind, by the action of high energy radiation in the presence of photoinitiators. Useful polymerization initiators include all compounds which decompose into free radicals under the polymerization conditions, for example peroxides, hydroperoxides, hydrogen peroxides, persulfates, azo compounds and redox catalysts. The use of water-soluble initiators is preferred. In some cases it is advantageous to use mixtures of different polymerization initiators, for example mixtures of hydrogen peroxide and sodium peroxodisulfate or potassium peroxodisulfate. Mixtures of hydrogen peroxide and sodium peroxodisulfate may be used in any proportion. Examples of suitable organic peroxides are acetylacetone peroxide, methyl ethyl ketone peroxide, tert-butyl hydroperoxide, cumene hydroperoxide, tert-amyl perpivalate, tert-butyl perpivalate, tert-butyl perneohexanoate, tert-butyl perisobutyrate, tert-butyl per-2-ethylhexanoate, tert-butyl perisononanoate, tert-butyl permaleate, tert-butyl perbenzoate, di(2-ethylhexyl) peroxydicarbonate, dicyclohexyl peroxydicarbonate, di(4-tert-butylcyclohexyl) peroxydicarbonate, dimyristyl peroxydicarbonate, diacetyl peroxydicarbonate, allyl peresters, cumyl peroxyneodecanoate, tert-butyl per-3,5,5-trimethylhexanoate, acetylcyclohexylsulfonyl peroxide, dilauryl peroxide, dibenzoyl peroxide and tert-amyl perneodecanoate. Particularly suitable polymerization initiators are water-soluble azo initiators, e.g., 2,2′-azobis(2-amidinopropane)dihydrochloride, 2,2′-azobis(N,N′-dimethylene)isobutyramidine dihydrochloride, 2-(carbamoylazo)isobutyronitrile, 2,2′-azobis[2-(2′-imidazolin-2-yl)propane]dihydrochloride and 4,4′-azobis(4-cyanovaleric acid). The polymerization initiators mentioned are used in customary amounts, for example in amounts of from 0.01 to 5%, preferably from 0.05 to 2.0%, by weight, based on the monomers to be polymerized.

Useful initiators also include redox catalysts. In redox catalysts, the oxidizing component is at least one of the above-specified per compounds and the reducing component is for example ascorbic acid, glucose, sorbose, ammonium or alkali metal bisulfite, sulfite, thiosulfate, hyposulfite, pyrosulfite or sulfide, or a metal salt, such as iron(II) ions or sodium hydroxymethylsulfoxylate. The reducing component in the redox catalyst is preferably ascorbic acid or sodium sulfite. Based on the amount of monomers used in the polymerization, from 3×10⁻⁶ to 1 mol % may be used for the reducing component of the redox catalyst system and from 0.001 to 5.0 mol % for the oxidizing component of the redox catalyst, for example.

When the polymerization is initiated using high energy radiation, the initiator used is customarily a photoinitiator. Photoinitiators include for example α-splitters, H-abstracting systems or else azides. Examples of such initiators are benzophenone derivatives such as Michler's ketone, phenanthrene derivatives, fluorene derivatives, anthraquinone derivatives, thioxanthone derivatives, coumarin derivatives, benzoin ethers and derivatives thereof, azo compounds such as the above-mentioned free-radical formers, substituted hexaarylbisimidazoles or acylphosphine oxides. Examples of azides are: 2-(N,N-dimethylamino)ethyl 4-azidocinnamate, 2-(N,N-dimethylamino)ethyl 4-azidonaphthyl ketone, 2-(N,N-dimethylamino)ethyl 4-azidobenzoate, 5-azido-1-naphthyl 2′-(N,N-dimethylamino)ethyl sulfone, N-(4-sulfonylazidophenyl)maleimide, N-acetyl-4-sulfonylazidoaniline, 4-sulfonylazidoaniline, 4-azidoaniline, 4-azidophenacyl bromide, p-azidobenzoic acid, 2,6-bis(p-azidobenzylidene)cyclohexanone and 2,6-bis(p-azidobenzylidene)-4-methylcyclohexanone. Photoinitiators, if used, are customarily used in amounts of from 0.01 to 5% of the weight of the monomers to be polymerized.

The crosslinked polymers are preferably used in partially neutralized form. The degree of neutralization is generally in the range from 5 to 80%, in the case of neutral superabsorbents preferably 60-80 mol %, in the case of acidic superabsorbents preferably in the range from 5 to 60 mol %, more preferably in the range from 10 to 40 mol %, particularly preferably in the range from 20 to 30 mol %, based on the monomers containing acid groups. Useful neutralizing agents include alkali metal bases or ammonia/amines. Preference is given to the use of aqueous sodium hydroxide solution, aqueous potassium hydroxide solution or aqueous lithium hydroxide solution. However, neutralization may also be effected using sodium carbonate, sodium bicarbonate, potassium carbonate or potassium bicarbonate or other carbonates or bicarbonates or ammonia. Moreover primary, secondary and tertiary amines may be used.

Alternatively, the degree of neutralization can be set before, during or after the polymerization in all apparatuses suitable for this purpose. The neutralization can be effected for example directly in a kneader used for the polymerization.

Industrial processes useful for making these products include all processes which are customarily used to make superabsorbers, as described for example in Chapter 3 of “Modern Superabsorbent Polymer Technology”, F. L. Buchholz and A. T. Graham, Wiley-VCH, 1998.

Polymerization in aqueous solution is preferably conducted as a gel polymerization. It involves 10-70% strength by weight aqueous solutions of the monomers and optionally of a suitable grafting base being polymerized in the presence of a free-radical initiator by utilizing the Trommsdorff-Norrish effect.

The polymerization reaction may be carried out at from 0 to 150° C., preferably at from 10 to 100° C., not only at atmospheric pressure but also at superatmospheric or reduced pressure. As is customary, the polymerization may also be conducted in a protective gas atmosphere, preferably under nitrogen.

By subsequently heating the addition polymer gels at from 50 to 130° C., preferably at from 70 to 100° C., the quality characteristics of the addition polymers can be further improved.

The acrylic acid used in the manufacture of superabsorbents is generally stabilized with phenolic compounds, preferably p-methoxyphenol (Modern Superabsorbent Polymer Technology, John Wiley & Sons, Inc., 1998, Chapter 2.2.2.1 and Chapter 2.5.3). The inhibitor has to be rendered harmless prior to the free-radical polymerization. This is generally accomplished by inertizing with nitrogen or carbon dioxide. p-Methoxyphenol is known to require oxygen for effective stabilization. After the polymerization has ended, however, the inhibitor remains in the product.

The acrylic acid used for manufacturing the absorbent resins generally has the following composition:

| | |
|---|---|
| Acrylic acid | 99.5-99.95% by weight |
| Acetic acid | 0.01-0.5% by weight |
| Propionic acid | 0.001-0.1% by weight |
| Diacrylic acid | 0.005-0.2% by weight |
| Aldehydes | max. 5 ppm |
| Inhibitor | 150-250 ppm |
| (e.g. p-methoxyphenol) | |

Corresponding concentrations apply to other acid-functional monomers. The acrylic acid can be produced by any desired method.

c) Surface Postcrosslinking

Hydrogel-forming polymers which are surface postcrosslinked are preferred. Surface postcrosslinking may be carried out in a conventional manner using dried, ground and classified polymer particles.

Compounds capable of reacting with the functional groups of the polymers by crosslinking are applied for this purpose to the surface of the hydrogel particles, preferably in the form of an aqueous solution. The aqueous solution may contain water-miscible organic solvents. Suitable solvents are alcohols such as methanol, ethanol, i-propanol ethylene glycol, propylene glycol or acetone.

The subsequent crosslinking reacts polymers which have been prepared by the polymerization of the above-mentioned monoethylenically unsaturated acids and optionally monoethylenically unsaturated comonomers and which have a molecular weight of greater than 5 000, preferably greater than 50 000, with compounds which have at least two groups reactive toward acid groups. This reaction can take place at room temperature or else at elevated temperatures up to 220° C.

Suitable postcrosslinkers include for example:
di- or polyglycidyl compounds such as diglycidyl phosphonates or ethylene glycol diglycidyl ether, bischlorohydrin ethers of polyalkylene glycols,
alkoxysilyl compounds,
polyaziridines, aziridine compounds based on polyethers or substituted hydrocarbons, for example bis-N-aziridinomethane,
polyamines or polyamidoamines and their reaction products with epichlorohydrin,
polyols such as ethylene glycol, 1,2-propanediol, 1,4-butanediol, glycerol, methyltriglycol, polyethylene glycols having an average molecular weight M_{w} of 200-10 000, di- and polyglycerol, pentaerythritol, sorbitol, the ethoxylates of these polyols and their esters with carboxylic acids or carbonic acid such as ethylene carbonate or propylene carbonate,
carbonic acid derivatives such as urea, thiourea, guanidine, dicyandiamide, 2-oxazolidinone and its derivatives, bisoxazoline, polyoxazolines, di- and polyisocyanates,
di- and poly-N-methylol compounds such as, for example, methylenebis(N-methylolmethacrylamide) or melamine-formaldehyde resins,
compounds having two or more blocked isocyanate groups such as, for example, trimethylhexamethylene diisocyanate blocked with 2,2,3,6-tetramethylpiperidin-4-one.

If necessary, acidic catalysts may be added, for example p-toluenesulfonic acid, phosphoric acid, boric acid or ammonium dihydrogenphosphate.

Particularly suitable postcrosslinkers are di- or polyglycidyl compounds such as ethylene glycol diglycidyl ether, the reaction products of polyamidoamines with epichlorohydrin and 2-oxazolidinone.

The crosslinker solution is preferably applied by spraying with a solution of the crosslinker in conventional reaction mixers or mixing and drying equipment such as Patterson-Kelly mixers, DRAIS turbulence mixers, Lödige mixers, screw mixers, plate mixers, fluidized bed mixers and Schugi Mix, for example. The spraying of the crosslinker solution may be followed by a heat treatment step, preferably in a downstream dryer, at from 80 to 230° C., preferably 80-190° C., particularly preferably at from 100 to 160° C., for from 5 minutes to 6 hours, preferably from 10 minutes to 2 hours, particularly preferably from 10 minutes to 1 hour, during which not only cracking products but also solvent fractions can be removed. But the drying may also take place in the mixer itself, by heating the jacket or by blowing in a preheated carrier gas.

In a particularly preferred embodiment of the invention, the hydrophilicity of the particle surface of the hydrogel-forming polymer is additionally modified by formation of complexes. The formation of complexes on the outer shell of the hydrogel particles is effected by spraying with solutions of divalent or more highly valent metal salt solutions, and the metal cations can react with the acid groups of the polymer to form complexes. Examples of divalent or more highly valent metal cations are Mg²⁺, Ca²⁺, Al³⁺, Sc³⁺, Ti⁴⁺, Mn²⁺, Fe^{2+/3+}, Co²⁺, Ni²⁺, Cu^{+/2+}, Zn²⁺, Y³⁺, Zr⁴⁺, Ag⁺, La³⁺, Ce⁴⁺, Hf⁴⁺, and Au^{+/3}+, preferred metal cations are Mg²⁺, Ca²⁺, Al³⁺, Ti⁴⁺, Zr⁴⁺ and La³⁺, and particularly preferred metal cations are Al³⁺, Ti⁴⁺ and Zr⁴⁺. The metal cations may be used not only alone but also mixed with each other. Of the metal cations mentioned, all metal salts are suitable that possess adequate solubility in the solvent to be used. Of particular suitability are metal salts with weakly complexing anions such as for example chloride, nitrate and sulfate. Useful solvents for the metal salts include water, alcohols, DMF, DMSO and also mixtures thereof. Particular preference is given to water and water/alcohol mixtures such as for example water-methanol or water-1,2-propanediol.

The spraying of the metal salt solution onto the particles of the hydrogel-forming polymer may be effected not only before but also after the surface postcrosslinking of the particles. In a particularly preferred process, the spraying of the metal salt solution takes place in the same step as the spraying with the crosslinker solution, the two solutions being sprayed in succession or simultaneously via two nozzles or the crosslinker and metal salt solutions may be sprayed conjointly through a single nozzle.

Optionally, the hydrogel-forming polymers may be further modified by admixture of finely divided inorganic solids, for example silica, alumina, titanium dioxide and iron(II) oxide, to further augment the effects of the surface aftertreatment. Particular preference is given to the admixture of hydrophilic silica or of alumina having an average primary particle size of from 4 to 50 nm and a specific surface area of 50-450 m²/g. The admixture of finely divided inorganic solids preferably takes place after the surface modification through crosslinking/complexing, but may also be carried out before or during these surface modifications.

Properties of the hydrogel-forming polymers according to the invention, of the method of making and of the acrylic acid used.

The inventive hydrogel-forming polymers capable of absorbing aqueous fluids combine a high ultimate absorption capacity with high gel strength and permeability and also high retention.

The SFC value [in 10⁻⁷ cm³s/g] of the inventive hydrogel-forming polymers as is measurable by the methods indicated in the description part, is preferably more than 1, especially 2, 4, 6, 8, 10, 12, 14, 16, 18, 20 or higher, more preferably 22 especially 24, 26, 28, 30, 32 or higher.

The CRC value [g/g] of the inventive hydrogel-forming polymers, as is measurable by the methods indicated in the description part, is preferably more than 15, especially 16, 18, 20, 22, 24, or higher, more preferably 25, especially 26, 27, 28, 29, 30, 31, 32 or higher.

The AUL 0.7 psi value [g/g] of the inventive hydrogel-forming polymers, as is measurable by the methods indicated in the description part, is preferably more than 4, especially 6, 8, 10, 12, or higher, particularly preferably 13 especially 14, 15, 16, 17, 18, 19, 20, 21, 22, 23, 24, 25, 26, 27, 28, or higher.

Particular preference is given to a combination of the threshold values, for example SFC with CRC, SFC with AUL, AUL with CRC, especially to triple combinations of SFC, AUL and CRC.

Polymers based on polyacrylate refers herein to such hydrogel-forming polymers capable of absorbing aqueous fluids that contain at least polyacrylates. In the case of graft polymers, it is preferably polyacrylate which has been grafted on. The fraction of acrylic acid as a hydrophilic monomer in (co)polymerized polymers or in graft (co)polymers is preferably 50% by weight or more, preferably 80% by weight or more; more preferably more than 90% by weight, 95% by weight, 98% by weight, especially 99% by weight and more.

Tocopherol refers to compounds of the following formula: 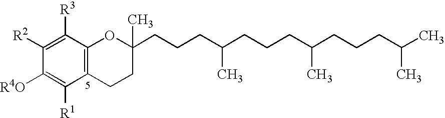
where R¹ is H or methyl, R² is H or methyl, R³ is H or methyl and R⁴ is H or an acid radical of 1-20 carbon atoms.

Preferred radicals for R⁴ are acetyl, ascorbyl, succinyl, nicotinyl and other physiologically acceptable carboxylic acids. The carboxylic acids can be mono-, di- or tricarboxylic acids.

Preference is given to alpha-tocopherol where R1=R2=R3=methyl, especially racemic α-tocopherol. R4 is particularly preferably H or acetyl. Particular preference is given to RRR-alpha-tocopherol.

Tocopherol is present in the polymer and in the acrylic acid monomer in a concentration which is preferably in the range from 10-1000 ppm, preferably in the range from 50-500 ppm, and especially in the range from 100-300 ppm, based on acid-functional monomers, especially on acrylic acid, or on acid units in the polymer.

Preference is given to acid-functional monomer, especially acrylic acid, containing essentially only tocopherol stabilizer.

Essentially it is to be understood as meaning that tocopherol, in terms of the mol % of the stabilizers, has the highest fraction, preferably more than 90 mol %, especially more than 95, 96, 97, 98, 99 mol %. In free-radical polymerization, α-tocopherol is advantageous over p-methoxyphenol in that the free-radical polymerization is quicker to light off and proceeds more smoothly. The products obtained are less yellowish and have a Z % value of more than 70, especially more than 75, as measured using a Hunterlab LS 5100 calorimeter. The induction period in the inventive process, involving acrylic acid for example, is ≦20 sec, especially ≦15 sec, and hence distinctly shorter than on addition of MEHQ.

EP 449 913 (U.S. Pat. No. 5,159,106) describes a process for preparing (meth)acrylic esters of polyhydric alkanols by an acid-catalyzed esterification reaction in the presence of tocopherols. This is said to prevent discoloration of the esterification products.

WO 99/01410 (EP 998 437) recommends using alpha-tocopherol as a polymerization inhibitor in the production, storage and transportation of vinyl monomers, preferably acrylonitrile.

U.S. Pat. No. 5,461,124 describes the use of tocopherol in the manufacture of surgical adhesives and surgical cement.

Deployment and Use of Hydrogel-Forming Polymers

The present invention further provides for the use of the abovementioned hydrogel forming polymers in hygiene articles comprising
(A) a liquid pervious topsheet
(B) a liquid impervious backsheet
(C) a core positioned between (A) and (B) and comprising
   (C1) 10-100% by weight of the hydrogel forming polymer according to the invention
   (C2) 0-90% by weight of hydrophilic fiber material
(D) optionally a tissue layer positioned directly above and below said core (C) and
(E) optionally an acquisition layer positioned between (A) and (C).

Hygiene articles for the purposes of the present invention include not only incontinence pads and incontinence briefs for adults but also diapers for infants.

The liquid pervious topsheet (A) is the layer which is in direct contact with the skin of the wearer. Its material comprises customary synthetic or manufactured fibers or films of polyesters, polyolefins, rayon or natural fibers such as cotton. In the case of non-woven materials the fibers are generally joined together by binders such as polyacrylates. Preferred materials are polyesters, rayon or blends thereof, polyethylene and polypropylene.

The liquid impervious layer (B) is generally a sheet of polyethylene or polypropylene.

The core (C) includes not only the hydrogel forming polymer (C1) of the invention but also hydrophilic fiber material (C2). By hydrophilic is meant that aqueous fluids spread quickly over the fiber. The fiber material is usually cellulose, modified cellulose, rayon, polyester such as polyethylene terephthlate. Particular preference is given to cellulose fibers such as pulp. The fibers generally have a diameter of 1-200 μm, and preferably 10-100 μm, and also have a minimum length of 1 mm.

The fraction of hydrophilic fiber material based on the total amount of the core is preferably 20-80% by weight and particularly preferably 40-70% by weight.

Diaper construction and shape is common knowledge and described for example in EP-A-0 316 518 and EP-A-0 202 127. Diapers and other hygiene articles are generally also described in WO 00/65084, especially at pages 6-15, WO 00/65348, especially at pages 4-17, WO 00/35502, especially pages 3-9, DE 19737434, WO 98/8439. These references and the references therein are hereby expressly incorporated herein.

The acidic hydrogel-forming polymers of the invention are very useful as absorbents for water and aqueous fluids, so that they may be used with advantage as a water retainer in market gardening, as a filter aid and particularly as an absorbent component in hygiene articles such as diapers, tampons or sanitary napkins.

Experimental Part

Test Methods

a) Centrifuge Retention Capacity (CRC)

This method measures the free swellability of the hydrogel in a teabag. 0.2000±0.0050 g of dried hydrogel (particle size fraction 106-850 μm) are weighed into a teabag 60×85 mm in size which is subsequently sealed. The teabag is placed for 30 minutes in an excess of 0.9% by weight sodium chloride solution (at least 0.83 l of sodium chloride solution/1 g of polymer powder). The teabag is then centrifuged for 3 minutes at 250 g. The amount of liquid is determined by weighing back the centrifuged teabag.

b) Absorbency Under Load (AUL) (0.7 psi)

The measuring cell for determining AUL 0.7 psi is a Plexiglass cylinder 60 mm in internal diameter and 50 mm in height. Adhesively attached to its underside is a stainless steel sieve bottom having a mesh size of 36 μm. The measuring cell further includes a plastic plate having a diameter of 59 mm and a weight which can be placed in the measuring cell together with the plastic plate. The plastic plate and the weight together weigh 1 345 g. AUL 0.7 psi is determined by determining the weight of the empty Plexiglass cylinder and of the plastic plate and recording it as W₀. 0.900±0.005 g of hydrogel-forming polymer (particle size distribution 150-800 μm) is then weighed into the Plexiglass cylinder and distributed very uniformly over the stainless steel sieve bottom. The plastic plate is then carefully placed in the Plexiglass cylinder, the entire unit is weighed and the weight is recorded as Wₐ. The weight is then placed on the plastic plate in the Plexiglass cylinder. A ceramic filter plate 120 mm in diameter and 0 in porosity is then placed in the middle of a Petri dish 200 mm in diameter and 30 mm in height and sufficient 0.9% by weight sodium chloride solution is introduced for the surface of the liquid to be level with the filter plate surface without the surface of the filter plate being wetted. A round filter paper 90 mm in diameter and <20 μm in pore size (S&S 589 Schwarzband from Schleicher & Schüll) is subsequently placed on the ceramic plate. The Plexiglass cylinder containing hydrogel-forming polymer is then placed with plastic plate and weight on top of the filter paper and left there for 60 minutes. At the end of this period, the complete unit is removed from the Petri dish and subsequently the weight is removed from the Plexiglass cylinder. The Plexiglass cylinder containing swollen hydrogel is weighed together with the plastic plate and the weight recorded as W_{b}.

AUL was calculated by the following equation:
AUL 0.7 psi [g/g]=[W_{b}−Wₐ]/[Wₐ−W₀]
c) Saline Flow Conductivity (SFC)

The test method for determining SFC is described in U.S. Pat. No. 5,599,335.

### EXAMPLES

### Example of SAP Production

Starting from 1 735 g of acrylic acid, admixed with racemic α-tocopherol or MEHQ, 1 445 g of 50% aqueous sodium hydroxide solution and 2 760 g of water, and approximately 30% sodium acrylate solution was prepared in a conventional manner and deoxygenated with countercurrent nitrogen in a stripping column in a conventional manner.

The substantially oxygen-free solution was transferred into a Werner & Pfleiderer LUK 8 trough kneader, and mixed with 7.8 g of polyethylene glycol diacrylate and thoroughly mixed through. The reactor was blanketed with nitrogen throughout the entire reaction time.

The initiator system, initially 32.12 g of sodium persulfate (15% solution) and then 20.79 g of ascorbic acid (0.5%), was added while the stirrer shafts were in motion. On completion of the addition, the contents of the kneader were heated at a heating fluid temperature of 74° C. The mixture began to warm up and became viscid (induction period). As soon as the maximum polymerization temperature was exceeded, the heating was switched off and a supplementary polymerization was carried out for about 15 minutes. The contents of the kneader were cooled down to 50-60° C. and discharged onto a drying sieve to form a thin layer and dried in a drying cabinet at 160° C. for about 90 minutes. The dried powder was subsequently adjusted to a final particle size of from 100 to 850 μm by grinding and sieving.

Surface Crosslinking

A 5 l capacity Lödige plowshare mixer was charged with 1.8 kg of superabsorbent powder prepared as per the above prescription. A solution of 1.4 g of ethylene glycol diglycidyl ether, 59 g of water and 29 g of 1,2-propanediol was sprayed onto the powder in the course of from 5 to 10 min. The product is raised to a temperature of 120° C. and held at that temperature for 60 minutes in order that the solvent may be distilled off again. This is followed by cooling before the product is discharged and sieved to the particle size fraction 100-850 μm.

### 1st Example

Acrylic acid with 250 ppm of racemic α-tocopherol
Induction period≦10 sec
End product very white (Z % value: 76)^{*)}
   *) Color determination (Z % values) using Hunterlab LS 5100 calorimeter

### 2nd Example

Acrylic acid with 220 ppm MEHQ
Induction period 70 sec
End product slightly yellow to yellow (Z % value: 63)^{*)}
   *) Color determination (Z % values) using Hunterlab LS 5100 calorimeter
Induction period—α-tocopherol: typically 5-15 sec MEHQ: typically 50-150 sec

1.-12. (Cancelled)

13. A hydrogel-forming polymer capable of absorbing an aqueous fluid and based on an acid-functional polymer containing tocopherol.

14. The hydrogel-forming polymer of claim 1 wherein the acid-functional polymer is a polyacrylate.

15. The hydrogel-forming polymer of claim 1 wherein the tocopherol is distributed over the polymer.

16. The hydrogel-forming polymer of claim 1 wherein the tocopherol is alpha-tocopherol.

17. The hydrogel-forming polymer of claim 1 wherein the tocopherol is present in an amount from 10 to 1,000 ppm, based on the acid-functional monomer.

18. A method of preparing a hydrogel-forming polymer capable of absorbing an aqueous fluid and based on an acid-functional polymer comprising using an acid-functional monomer that contains tocopherol.

19. The method of claim 18 wherein the acid-functional monomer comprises acrylic acid, and wherein the acrylic acid contains at least 50 ppm of tocopherol, and an induction period is at most 20 seconds.

20. The method of claim 18 wherein the acid-functional monomer comprises acrylic acid and wherein the acrylic acid contains at least 50 ppm of tocopherol, and a Z % value of the hydrogel-forming polymer is greater than 70.

21. A method of preparing a hydrogel-forming polymer capable of absorbing an aqueous fluid comprising the use of an acid-functional monomer, wherein the acid-functional monomer contains tocopherol.

22. The method of claim 21 wherein the tocopherol comprises alpha-tocopherol.

23. The method of claim 21 wherein the acid-functional monomer contains tocopherol as a sole stabilizer.

24. The method of claim 21 wherein the acid-functional monomer comprises acrylic acid or an acrylate salt.

25. A hydrogel-forming polymer capable of absorbing aqueous fluids prepared by the method of claim 21.

26. A method of absorbing an aqueous fluid comprising contacting the aqueous fluid with a hydrogel-forming polymer of claim 1.

27. A hygiene article comprising a hydrogel-forming polymer of claim 1.
